(19) United States
(12) Patent Application Publication
(10) Pub. No.: US 2021/0248702 A1
(43) Pub. Date: Aug. 12, 2021

(54) Educational Delivery Platform

(71) Applicants: Liz Imperio, Los Angeles, CA (US); Chad Carlberg, Los Angeles, CA (US)

(72) Inventors: Liz Imperio, Los Angeles, CA (US); Chad Carlberg, Los Angeles, CA (US)

(21) Appl. No.: 16/972,679

(22) PCT Filed: Jun. 8, 2019
(86) PCT No.: PCT/US2019/036188

§ 371 Date: Dec. 7, 2020

Related U.S. Application Data
(60) Provisional application No. 62/682,528, filed on Jun. 8, 2018.

(51) Int. Cl. G06Q 50/20; G06Q 50/00; G06Q 10/10; G06Q 30/02; G06Q 20/12; A63F 13/79; H04N 21/475

(52) U.S. Cl. CPC G06Q 50/205; G06Q 50/01; G06Q 10/101; G06Q 30/0213; G06Q 30/0224; G16H 20/30; G06Q 30/0236; G06Q 20/127; G06Q 10/103; A63F 13/79; H04N 21/4758; G06Q 30/0226

(57) ABSTRACT

A computer implemented system and method for a gamified social media educational platform for interacting with a participant via the Internet using four distinct educational modules. An introduction module for focus and commitment. A setting goals module for long term, flexible direction in life. A success module for harnessing a participant's passion. A tools of the trade module for mental, emotional, physical tools. And an activity sessions module for selecting physical exercises for the participant.

### CROSS-REFERENCE TO RELATED APPLICATIONS

This application claims the benefit under 35 U.S.C. § 119(e) of U.S. Provisional Patent Application Ser. No. U.S. 62/682,528, filed on 2018 Jun. 8, the contents of which are incorporated herein by reference in their entirety.

### FIELD OF THE INVENTION

The present invention is in the technical field of education, and more particularly to system 100 for gamified social media educational system for both online and in person training for participants to take control of their lives.

### SUMMARY

The present invention overcomes the limitations of the prior art by providing a computer implemented method for a gamified social media educational platform. The method begins by first, executing instructions on a server with at least one processor and at least one storage for interacting with a participant via the Internet using four distinct educational modules. The four educational modules are an introduction module for focus and commitment, a setting goals module for long term, flexible direction in life, a success module for harnessing a participant's passion, and a tools of the trade module for mental, emotional, physical tools. Additional modules comprise instructions to determine studio activity sessions for an at home conditioning course, healthy living for mental and physical health, activity life training etiquette, and beyond the activity professions and educational opportunities based upon the participant's goal.

The introduction module comprises instructions presented to the participant for teaching basic habits required of every participant to maximize and elevate the experience and improve the end result and teaching fundamentals values need to complete the course. The introduction module comprises instructions presented to the participant for determining a value of long-term goals that have been set, determining a value of short-term goals that have been set, determining a reason to visualize the long-term and short-term goals, researching the long-term and short-term goals, organizing the long-term and short-term goals for the individual participant, determining a follow through plan, so that the participant continues to work, and displaying a workbook with the modules, where a participant builds a vision board to express the participant's ideal future. Additionally, the participant is shown compiled professional research to assist with their researching.

The success module presents the participant with daily steps for success, where each step is one day of an activity, determining investment in self, examples by other professionals in the field using personal examples so the participant can determine their connection to the activity, determining emotional connection to the activity, determining the participant's inspiration for the activity and how to stay inspired, teaching the participant how to understand the “why” in the activity, when there is a passion driving the activity, and filling out a daily journal of exercises provide by the system 100 to assist in maintaining the participant's goal that were previously determined.

The tools of the trade module has instructions for building the participant's self worth by introducing mental, physical and emotional tools for a participant to master, determining mental tools to assist the participant with the activity, determining physical tools, the building blocks of the activity, determining any advanced physical tools for the activity, emotional tools for interconnecting the participant's inner self and external connection to the activity, determining the participant's attitude and dealing with failure. Digital workbook with specific exercises for each of these steps, showing participants how to sharpen their unique skills and mindset, in order to achieve their long-term goals. There is also steps for addressing the mental, emotional and physical skills of the specific task the participant is pursuing.

Selecting physical exercises for the participant for the activity module comprises a deeper explanation of the level of conditioning needed by the participants, to keep their body safe.

The healthy living module comprises demonstrating physical injury prevention, determining nutrition for the participant, and evaluating mental health of the participant.

The activity life module comprises demonstrating activity etiquette, demonstrating communication between participant and parent, communicating with teachers and studio owners by the participant, finding a life, school and activity balance, providing methods for dealing with the anxiety, providing expectation analysis for rehearsals and practice for the participant to achieve the best results, and providing expectation analysis for getting the most out of performance for an activity.

The beyond the activity module comprises determining by the participant professional aspirations, determining by the participant educational aspirations, determining by the participant continued education and determining by the participant an overall understanding of what the participant has to offer beyond school.

There is also provided a computer enabled gamified social media educational platform system having a server, a plurality of participant electronic devices communicatively coupled, via Internet, to the server, each participant electronic device associated a plurality of participants, and a second storage operably connected to the server, wherein gamification instructions for learning executable on the server and on the plurality of participant electronic devices to motivate participants and to control interactions between the plurality of participants and a social media educational platform using their respective electronic devices. The server has at least one processor providing access to an educational platform, a plurality of educational modules, instructions for reading participant information, educational module instructions from a first storage medium, storage for storing the participant information, including: progress, scoring, bonuses, coins, badges, gems and other relevant data of the participant in the storage medium, a plurality of participant electronic devices communicatively coupled, via Internet, to the server, each participant electronic device associated a plurality of participants, and a second storage operably connected to the server, wherein gamification instructions for learning executable on the server and on the plurality of participant electronic devices to motivate participants and to control interactions between the plurality of participants and a social media educational platform using their respective electronic devices, wherein the respective electronic devices can display an introduction module for focus and commitment, a setting goals module for long term, flexible direction in life, a success module for harnessing a participant's passion, a tools of the trade module for mental, emotional, physical tools, and an activity sessions module for selecting physical exercises for the participant.

The gamified social media educational platform has certification courses for teachers, participants and parents, private coaching, wherein certified professionals give sixty minute coaching sessions to participants, professional critiques, wherein a participant can upload a video of themselves performing an activity from a class or the activity that is analyzed by certified staff, and the video is returned to the participant with comments, suggestions and where to focus for the participant to achieve the greatest improvement, and classes taught by professionals in the industry.

Participants can pay coins earned using the system for classes. Participants learn the from the class, then film themselves doing the activity that was taught. Then, the videos will be entered into a contest, where winners get more coins and professional critiques are available. A virtual-battle module will be available for videos entered above. Other participants vote on the virtual-battle videos to determine a winner. Additional benefits are provided to both the winner and the other participants of the virtual-battle. Coaching sessions, based on the uploaded videos, are also available online.

The system also has an activity wall module that provides participant's personal social media type wall, a group creation module, where a participant uses coins to create a group, join a group or both create and join a group, a list of active courses module to lead the participant back to the learning platform, a messaging and notifications module for communication from participants and staff, a real time chat module including video group conferencing, a friends/followers/following module that provides those social media functions, a coins module that tracks earned for daily logins, posts, and general activity within the system, wherein coins can be used to buy prizes in a reward center and can be purchased by signing up for a silver, gold, or platinum level monthly membership, a compliments module, wherein compliments can be sent by each participant, a commerce module, wherein participants pay to access another participants profile, and wherein a percentage of the sale is given to the participant who created the profile, a media module for that organizes all participant uploaded media, and a vendor module for selling gear related to the activity.

The system also has a reward center module, where coins earned by participants can be exchanged for other items, such as, groups, sending compliments, advanced social media features, classes, videos, certification courses, and gear.

A coins module, where participants earn coins by being involved in the system's social media, or by purchasing a membership level of silver, gold, or platinum membership. Coins are also awarded when a participant watches select videos, for taking surveys and winning contests. The badges module is activated after participants complete a purchase, watch a class, submit a video or submit homework, they earn a badge for their profile that is displayed on the participant's social media wall. There is also provided a gems module, where each badge earned by a participant is worth ten gems at the end of the month, the top ten gem earners win and get featured as “featured participant” on the membership site, increasing their exposure, wherein participants also earn gems when they receive a “compliment” from other participants.

The rewards module, provides participants with coins, badges and gems for getting involved in the social community, where the coins are redeemable for activity related merchandise, including videos of the teachings, classes, gear and additional social media perks such as video chats.

There is also provided a virtual-battle module that is a unique online competition between participants. The virtual-battle module provides that once an activity is learned in a class, the participant is prompted to record and download themselves performing the activity, which advances the participant to the next level. “The next step” where the participant creates the next activity and uploads it to the system and a professional critique is offered for the participant's video. Once uploaded, the participant is invited to a secret part of the system called “virtual-battle” where they are given levels of video challenges to upload and compete with other participants and wherein various prizes are awarded to the winner of each virtual-battle. Virtual-battle levels comprise participation with friends, at unique locations and/or special effects in editing software. Participants vote for their favorite in each virtual-battle and earn coins for voting. The participant with the most gems at the end of the month is featured on the main webpage, where gems are earned from badges in the learning center, or compliments in the social media platform.

The system also has a studio team module where participants sign up for an activity group to participate in competitive group learning. An owner of the activity group has a control panel so that the owner can: invite participants, manage groups and courses, track participant progress, schedule courses, topics and classes, broadcast announcements, archive round table discussions held with other group owners. Additionally, the control panel has a courses button that accesses participant certification, parent certification and assistant teaching training courses.

The parent certification course provides a video course with lessons, where the lessons are divided into smaller video modules covering at least all the sections listed below. Beginning with an introduction to the certification course. Then there is a focus on success lesson, for understanding that there is a different mindset than your used to focus on success vs. focus on things like the trophy. A finding balance lesson helps the parents understanding of the investment, the time and money spent on an activity, all sacrifices made by parents and participants. The injury prevention lesson focuses on keeping your child safe. The communication lessons develops skills so you can talk with a child, with your spouse, with your studio owner, etc. Then the importance of . . . lesson demonstrates how to, what to say, leaving emotions out. The supercharge success lesson shows parents about driving their child's passion for activity to greatness by harnessing passion. The performance anxiety deals with parent anxiety/participant anxiety, and how to overcome it. The self-worth lessons demonstrates building self-worth with the short term and long-term goals set, and understanding the value for the long term in each short term goal. The accountability lesson focuses on staying true to yourself first, staying true to your word and your commitment. They, 6, 7, 8 to Success lessons helps parents in creating their child's next step for their journey in the activity. How to use the lessons learned throughout the series to harness success in the activity, and in life. Finally, there are bonus lessons for training in social media, body image, college options, professional options, bullying, healthy living, and preparing for auditions.

### BACKGROUND

Currently, there is a gap with the education and training of young and professional level participants throughout the nation. For example, young competitive dancers, who feed the professional circuit, get good physical training with a well-rounded, ballet based foundation are missing many of the mental and emotional skills needed for success in dance and in life, when they make it to a professional level.

This problem exists in all sports and even activities such as chess and academics. participants are pushed to succeed at all costs excluding many social interactions and how to deal with life in general. A look at the scandals in both college and professional basketball reveals a problem that started years ago.

Most children participants don't want to take time to complete educational courses when it takes time or interferes with their passion. Also, there are many parents that push their children into activities to “make” them successful on the slim hopes of a professional career. This all consuming effort becomes the persons identity, and after their career ends, they don't always have any skills to fall back upon.

Therefore, there is a need for a gamified social media educational system 100 for both online and in person training for participants to take control of their lives.

### BRIEF DESCRIPTION OF THE DRAWINGS

These and other features, aspects and advantages of the present invention will become better understood with regard to the following description, appended claims, and accompanying figures where:

FIG. 1 is a flowchart diagram of a system 100 for a gamified social media educational system 100 for both online and in person training for participants to take control of their lives;

FIG. 2 is a screenshot of a participant's electronic device showing a home screen of the system 100 of FIG. 1; and

FIG. 3 is a flowchart diagram of some steps of a method for the system 100 of FIG. 1.

### DETAILED DESCRIPTION OF THE INVENTION

The present invention overcomes the limitations of the prior art by providing a gamified social media educational system 100 for both online and in person training for participants to take control of their lives.

All dimensions specified in this disclosure are by way of example only and are not intended to be limiting. Further, the proportions shown in these Figures are not necessarily to scale. As will be understood by those with skill in the art with reference to this disclosure, the actual dimensions and proportions of any system 100, any device or part of a system 100 or device disclosed in this disclosure will be determined by its intended use.

Methods and devices that implement the embodiments of the various features of the invention will now be described with reference to the drawings. The drawings and the associated descriptions are provided to illustrate embodiments of the invention and not to limit the scope of the invention. Reference in the specification to “one embodiment” or “an embodiment” is intended to indicate that a particular feature, structure, or characteristic described in connection with the embodiment is included in at least an embodiment of the invention. The appearances of the phrase “in one embodiment” or “an embodiment” in various places in the specification are not necessarily all referring to the same embodiment.

Throughout the drawings, reference numbers are re-used to indicate correspondence between referenced elements. In addition, the first digit of each reference number indicates the figure where the element first appears.

As used in this disclosure, except where the context requires otherwise, the term “comprise” and variations of the term, such as “comprising”, “comprises” and “comprised” are not intended to exclude other additives, components, integers or steps.

In the following description, specific details are given to provide a thorough understanding of the embodiments. However, it will be understood by one of ordinary skill in the art that the embodiments may be practiced without these specific details. Well-known circuits, structures and techniques may not be shown in detail in order not to obscure the embodiments. For example, circuits may be shown in block diagrams in order not to obscure the embodiments in unnecessary detail.

Also, it is noted that the embodiments may be described as a process that is depicted as a flowchart, a flow diagram, a structure diagram, or a block diagram. The flowcharts and block diagrams in the figures can illustrate the architecture, functionality, and operation of possible implementations of system 100s, methods and computer programs according to various embodiments disclosed. In this regard, each block in the flowchart or block diagrams can represent a module, segment, or portion of code, that can comprise one or more executable instructions for implementing the specified logical function(s). It should also be noted that, in some alternative implementations, the functions noted in the blocks may occur out of the order noted in the figures. Although a flowchart may describe the operations as a sequential process, many of the operations can be performed in parallel or concurrently. In addition, the order of the operations may be rearranged. A process is terminated when its operations are completed. A process may correspond to a method, a function, a procedure, a subroutine, a subprogram, etc. When a process corresponds to a function, its termination corresponds to a return of the function to the calling function or the main function. Additionally, each block of the block diagrams and/or flowchart illustration, and combinations of blocks in the block diagrams and/or flowchart illustration, can be implemented by special purpose hardware-based system 100s that perform the specified functions or acts, or combinations of special purpose hardware and computer instructions.

Moreover, a storage may represent one or more devices for storing data, including read-only memory (ROM), random access memory (RAM), magnetic disk storage mediums, optical storage mediums, flash memory devices and/or other non-transitory machine readable mediums for storing information. The term “machine readable medium” includes, but is not limited to portable or fixed storage devices, optical storage devices, wireless channels and various other non-transitory mediums capable of storing, comprising, containing, executing or carrying instruction(s) and/or data.

Furthermore, embodiments may be implemented by hardware, software, firmware, middleware, microcode, or a combination thereof. When implemented in software, firmware, middleware or microcode, the program code or code segments to perform the necessary tasks may be stored in a machine-readable medium such as a storage medium or other storage(s). One or more than one processor may perform the necessary tasks in series, distributed, concurrently or in parallel. A code segment may represent a procedure, a function, a subprogram, a program, a routine, a subroutine, a module, a software package, a class, or a combination of instructions, data structures, or program statements. A code segment may be coupled to another code segment or a hardware circuit by passing and/or receiving information, data, arguments, parameters, or memory contents. Information, arguments, parameters, data, etc. may be passed, forwarded, or transmitted through a suitable means including memory sharing, message passing, token passing, network transmission, etc. and are also referred to as an interface, where the interface is the point of interaction with software, or computer hardware, or with peripheral devices.

In the following description, certain terminology is used to describe certain features of one or more embodiments of the invention.

Various embodiments provide a gamified social media educational system 100 for both online and in person training for participants to take control of their lives. One embodiment of the present invention provides a gamified social media educational system 100 for both online and in person training for participants to take control of their lives. In another embodiment, there is provided a method for using the system 100. The system 100 and method will now be disclosed in detail.

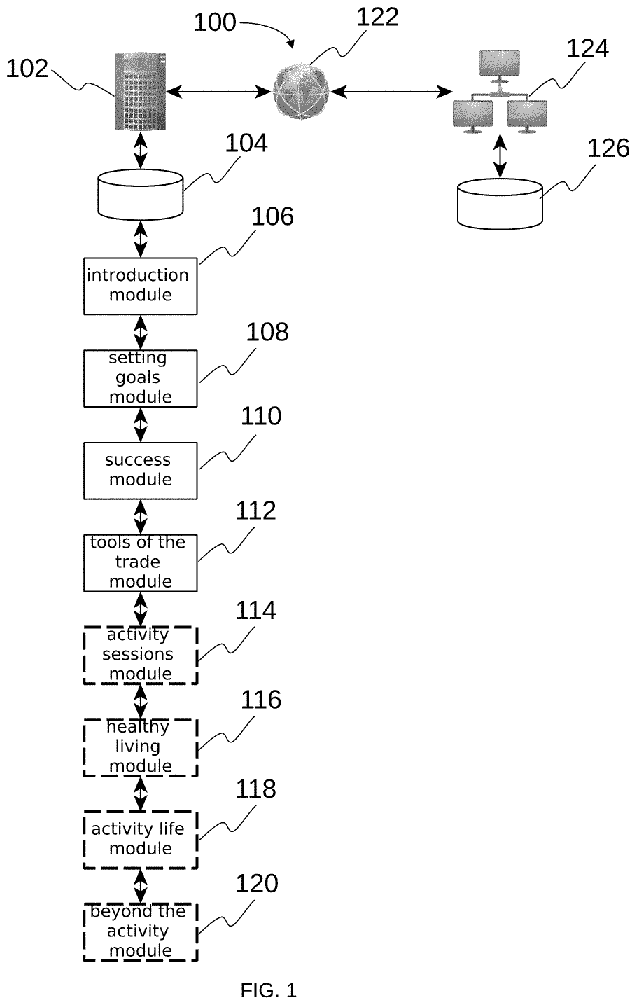

Referring now to FIG. 1, there is shown a flowchart diagram of a a gamified social media educational system 100 for both online and in person training for participants to take control of their lives. The system 100 encourages and rewards participants with “coins” “badges” and “gems” for getting involved in the social community, where the coins are redeemable for dance related merch, including videos of the teachings, classes, dance gear and additional social media perks such as video chats.

Gems (given for getting involved in the classes, receiving compliments from other participants, submitting videos and homework assignments, and generally for spending their coins) are collected in order to make the monthly leaderboard, where additional coins and gems can be earned.

This motivational software system 100 and the teachings themselves are unlike anything currently available in education.

The system 100 resulted from the observation that dancers, for example, simply aren't getting important life lessons anywhere else, and as a result, in general, are lacking major life skills that breed success. However, very few kids, nor their parents, were interested in bettering themselves, and even if they started gaining mass improvement in their lives as a result of taking a few classes, nearly none followed through, dancers nor parents. Therefore, the systems and methods disclosed herein were developed as a way to provide these lessons in a way that was both fun and sustainable. The system 100 and methods provide a “mental, spiritual and emotional” approach to an activity that breeds success in both the parents and the participants.

The system 100 adapts to each participants activity and can customize the approach taken with each participant to achieve their specific goals and overcome challenges. Additionally, parents can also participate and can continue the lessons after participation in person or online.

For example, in one embodiment the system 100 is used as an online dance convention. It's a social media site, similar to Facebook and Twitter, where participants are rewarded for interacting positively with one another (ie—Sending/receiving “compliments” from a list of options). Coins are received when others pay to view that participant's profile, and when that participant is generally being active in the site—posing pictures, updates, etc. So there's a really fun aspect to getting involved with their friends.

Coins can be redeemed for classes and certification programs, choreography challenges in a variety of genres, access to video archives, and advanced social media features. When these rewards are claimed, the participant earns badges that go on their profile, as well as gems. The top 10 gem earners of the month become the featured members, giving them visibility and exposure to the rest of their community.

The system 100 features dance classes, technique class, choreography, solo critiques and dance challenges like a live convention. only online.

The system 100 also comprises educational courses and one on one online coaching options available for dancers. Additionally, there are certifications program for dancers, parents, and teachers. Each of the courses focuses on the mental, emotional and spiritual aspects of the participant's passion. The system 100 is used to teach dancers how to harness their passion, and teach parents and teachers how to teach what is taught in the system 100.

The system 100 provides a location on the internet where kids who dance competitively, along with their parents & teachers, gather to discuss dance. They each have their own page, where they can interact with others, post messages, pictures and videos.

Participants gain coins when they are actively using the site or if they buy coins from the store. Coins are like money. they are used to buy prizes like educational courses, videos, private coaching, and dance gear. Participants also receive badges for completing courses, watching videos, or competing in dance based challenges. Whenever a participant earns a badge, they get a reward, such as, for example, ten “gems”. Gems are also earned when someone pays you a “compliment”, an icon and nicety chosen from a pre-designed list. The one with the most gems at the end of the month wins, and they get featured on the main web page.

The system 100 also comprises a dance competition called “Virtual-Battle”, that is a dance competition where dancers get creative with their craft and create videos based upon our various challenges, such as dance at a location, dance with your friends, or add a special effect to your video. The winners are the ones with the most votes, and win additional coins and gems for winning. Participants also earn the opportunity to be personally reviewed by professional affiliates to assist in the participant's pursuit to a professional career.

All these learning situations are tied together into a single platform. As will be understood by those will skill in the art with reference to this disclosure, the system 100 can be adapted for anything a participant has a passion for. The system 100 would allow any discipline to be customized. For example, the system 100 can be adapted for soccer players with a few minor changes, but still using the same educational platform, using this same formula, including the main lessons, for soccer instead of dance, or gymnastics, or wrestling, or football, or baseball.

What has been described is a new and improved system 100 and method for a gamified social media educational system 100 for both online and in person training for participants to take control of their lives, overcoming the limitations and disadvantages inherent in the related art.

The system 100 comprises instructions executable on a processor for interacting with a participant using four distinct modules. The modules comprise:

1) Introduction (Focus and Commitment)

2) Setting Goals—(long term, flexible direction in life)

3) Driving force of Success—(harnessing their passion)

4) Tools of the Trade (Mental, emotional, physical tools of a dancer)

5) Studio Sessions—(at home conditioning course)

6) Healthy Living (mental and physical health)

7) Activity Life (training etiquette)

8) Beyond the Activity (professions and educational opportunities)

In the introduction module 102 the system 100 covers the basic habits that are required in the practice of every dancer taking this course. This will maximizing and elevate the experience for the dancer as well as improve their end result. The fundamentals of where, why, and how to “Focus on success” sets up the dancer in a specific head space to focus on what matters. This is complimented by understanding the value of “Commitment”. Teaching our youth the practice of follow through, accountability and responsibility is a staple on our society that is losing its way. This Introduction better prepares the individual to take this course seriously and thoughtfully.

In the “Setting Goals” module 104, the system 100 displays steps to understanding where each step is one day. First, the value of goals is determined. This is an understanding of why it's important to have long term goals, even at a young age, and why short-term goals are important to support the long-term goal. Next, methods and reasons to visualize goals are determined. Then, how to research and why are determined. Next, how to organize and why are determined for the individual participant. Then, follow through is determined so that the participant continues to work the system 100. Optionally, as a bonus, the system 100 can display research that has been compiled for the professional area that the participant is concentrating on. A workbook is displayed along with the lessons, where participants build a vision board one day at a time. The vision board is complete by day five and expresses the participant's ideal future.

In the “Driving Force of your Success” module 106, the system 100 addresses the steps, where each step is one day, for success. First, the participant determines the investment in self. Next, the participant's connection to the activity is expressed by other professionals in the field using personal examples is determined. Then, the emotional connection to the activity is determined. This provides a stronger understanding of why the participants do what they do for the activity. Next, the system 100 helps the participant determine their particular inspiration and how to stay inspired. Finally, the system 100 teaches the participant an understanding of the importance of finding the “why” in anything you do, especially when there's a passion driving the activity. The system 100 provides a journal of exercises for the participant to fill out each day, helping them understand themselves better and maintaining the previously determined goals.

In the “Tools of the Trade” module 108, the system 100 uses a seven day course to address the mental physical and emotional tools a participant has to master. Additionally, this module focuses the participant on building self worth. First, the tools are introduced to the participant. Next, mental tools to assist the participant are determined. Then, physical tools, the building blocks of the activity are determined. Next, any advanced physical tools for the activity are determined. Then, emotional tools for interconnecting the participant's inner self and external connection to the activity. Next, attitude and dealing with failure are determined. Finally, the system 100 focuses the participant on self-worth and and understanding based on how the previous steps on how to best build self-worth. This module is designed to discuss the particulars of the activity, and the specific skills needed for this pursuit. Optionally, the module can address the mental, emotional and physical skills of the specific task the participant is pursuing. A digital workbook with specific exercises come along with each of these lessons, showing participants how to sharpen their unique skills and mindset, in order to achieve their long-term goals.

The “Activity Sessions” module presents the physical exercises, like a workout video, with a deeper explanation of the level of conditioning needed by the participants, to keep their body safe. In Studio Sessions, we address the physical training and have the kids warm up with an instructor in a DVD (home workout type program), as the instructor also points out where the focus should be on the body with each exercise in order to get the most out of it. “advanced conditioning”.

Additionally, the system 100 can comprise a “Healthy Living” module. The module comprises:
a. Physical injury prevention
b. Nutrition for the dancer
c. Mental Health

The system 100 further comprises a “Activity Life” module. The module includes:
a. Activity Etiquette
b. Communication between dancer and parent
c. Communication with Teachers and Studio Owners
d. Balance—Getting a better understanding about how to find balance in a pursuit that takes up all their time, with life and school.
e. Auditions—dealing with the anxiety and getting the most out of the audition process.
f. Rehearsals—expectations of a dancer, getting the most out of rehearsals
g. Performance—Getting the most out of performance

In the “Beyond the Activity” module the participant determines:
a. Professional aspirations
b. Educational aspirations
c. Continued education
d. “The Next Step”—an overall understanding of what the participant has to offer beyond school. At the end of each topic, a quiz comprised of multiple answers and essay questions are given before awarding them gems and a badge. This also allows the next section to be unlocked.

Once complete, the participant will have earned a certification and right to use the system 100's certification course on their resume.

There is also provided a method for a course for parents called “Parent Certification Course”, where the lessons are divided into ten modules. Once a parent completes the entire program, they will be “certified”, which gives them completion of a potential studio requirement.

The fifteen hour video course has ten modules and their sub-sections comprise the following:
1—Introduction to the Certification Course—new
2—Focus on SUCCESS—understanding a different mindset than your used to
3—Finding Balance—Understanding the investment: the time and money spent
4—Injury Prevention—keeping your kid safe
5—Communication—with child, with your spouse, with your studio owner
6—Supercharge Success—driving their child's passion for dance to greatness
7—Performance Anxiety—dealing with parent anxiety/dancer anxiety
8—Self-worth—Building self-worth with the long-term goals they set.
9—Accountability—staying true to yourself first.
10—5, 6, 7, 8 to Success—creating their dancer's next step for their journey in dance.

There is also provided a method for a “Teacher Certification Course”, that comprises several modules for teachers and is marketed to studio owners:
1) Learning Platform—Courses from above, plus videos, coaching, & classes for dancers.
2) Social Media—Facebook/Twitter type wall for dance community
3) Rewards system 100—Redeemable coins, badges, certificates and Gems rewarded for activities.
4) Competitions—Video submissions, per a unique set of rules (Virtual-Battle)
5) Stores—Membership store and affiliate vendor profile stores.

Each of the five modules also comprise sub-modules that make up the whole and tie the program together. A detailed description of all the modules is presented herein.

Learning Platform
1—Certification courses for teachers, participants and parents (Described in detail above)
2—Private Coaching—certified professionals give sixty minute coaching sessions to customers. The sessions are also available online via GoToWebinar and other live video sharing means. Participants will each have a custom page with a recording of their session and any notes/homework given during the session.
3—Professional Critiques—a participant can upload a video of themselves performing an activity from a class or the activity. The video is analyzed certified staff, and the video is sent back to participant with their professional comments, suggestions and where to focus for their individual greatest improvement.
4—Classes—Classes taught by some of the top professionals in the industry:
   a. participants pay coins for the class, learn the from the class, then film themselves doing the activity that was taught.
   b. participants are offered a link to purchase the music if it is part of the activity.
   c. Selected videos will be put into a contest, where winners get more coins. Professional critiques are available. Participant's enter the contest only after they upload in Virtual-Battle.
   d. Once uploaded, participants will be asked to create a video to finish “The next Step” and upload that video to the site for future competition.
   e. Once participants upload their video in step d, a Virtual-Battle module will become available for them to enter video contests.
5—Videos—Behind the scenes, interviews, parent Q & As, motivational videos, etc. can be created to help motivate the participant.

Social Media

1—Activity Wall—participants personal social media type wall.

2—Groups—for 200 coins a participant can create a group of their own or select from preloaded groups.
3—Courses—a list of active courses leads them back to the Learning Platform. When participants earn a badge for completing a section, its uploaded to the social media module to show achievement and encourage others to join.
4—Messages—messages and notifications from members and staff. Can be customized by participants.
5—Chat feature—Mobile app available for download or use the desktop system 100. Participants chat in real time. Bonus features available in the Reward Center include video group conferencing and single player games to name a few.
6—Friends/Followers/Following—standard social media terms.
7—Coins—earned for daily logins, posts, and general activity within the site. coins can be used to buy “prizes” in the Reward Center and can be purchased by signing up for a Silver, Gold, or Platinum level monthly membership.
8—Compliments—participants can send each other previously loaded compliments from a list, adding their own message to the compliment.
9—Participant commerce—Participants pay to access another participant's profile. A percentage of the sale is given to the participant who created the profile. Creating/Joining Groups, work in a similar fashion where part of the profit is shared with the group creator and admin.
10—Media—a bonus feature that organizes all media uploaded by the participant.
11—Outside vendor Stores—for selling gear related to the activity.

Reward System 100
1—Reward Center—coins earned by participants can be spent on things like groups, sending compliments, advanced social media features, classes, videos, certification courses, and gear.
2—Coins—members earn coins by being involved in the social media site, or by purchasing a membership level of silver, gold, or platinum membership. Coins are also gained when a participant pays to view your activity wall.
3—Badges—when participants complete a purchase, watch a class, submit a video or submit homework, they earn a badge for their profile.
4—Gems—each badge is worth ten gems. At the end of the month, the top ten gem earners win and get featured as “featured dancers” on the membership site, increasing their exposure. Participants also earn gems when they receive a “compliment” from other participants.

Competitions

1—Virtual-Battle is a unique online competition between participants.
a. Once an activity is learned in a class, the participant is prompted to record and download themselves performing the activity, which advances the participant to the next level. A pro critique is offered for their video.
b. “The Next Step”—this is where the participant creates the next activity and uploads it to the site. A pro critique is offered for their video.
c. Once uploaded, the participant is now invited to a secret part of the system called “Virtual-Battle” where they are given levels of video challenges to upload and compete with other participants. Various prizes are awarded to the winner of each Virtual-Battle.

Virtual-Battle levels can comprise: participation with friends, at unique locations, and/or with special effects in editing software.

2—Voting—participants vote for their favorite participants and earn coins for voitng.

3—Gems—The participant with the most gems at the end of the month is featured on the main webpage. Gems gained from badges in the learning center, or compliments in the social media platform.

Affiliate Stores will be able to set up their own shop within the social media platform, giving the ability to allow related vendors to set up a shop as their profile. A percentage of earnings are shared.

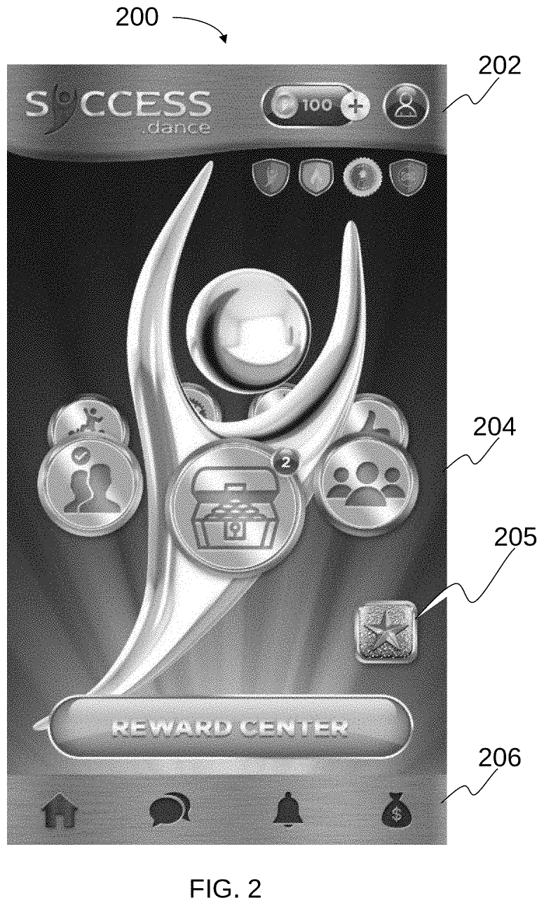

Referring now to FIG. 2, there is shown a screenshot of a participant's electronic device showing a home screen of the system 100. As can be seen, the participant's electronic device has access to all the various educational modules in the system 100. The home screen is broken down into three distinct area: a top section 202, a middle section, and a bottom section 206. Each section accesses a grouped set of modules from the system 100.

The top section 202 comprises, icons that access a participant's coins, a menu that accesses the participant's profile and other information about the system 100, and the badges area of the system 100. Additionally, the top section 202 comprises customized emoji's for social media, chats and compliments that participants can purchase with their coins. Live events can also be purchased by participants. In order to make the system 100 more enjoyable, custom animations both 2D and 3D are used throughout.

The middle section comprises icons for accessing a rewards center module, a social media module, a voting module, a challenges module, a master class module, a studio team module, a coaching and critiques module.

Additionally, the middle section also comprises a star icon 205. The star icon 205 provides participant access to a leader board module, a members module, a featured participant module, and a sponsors module.

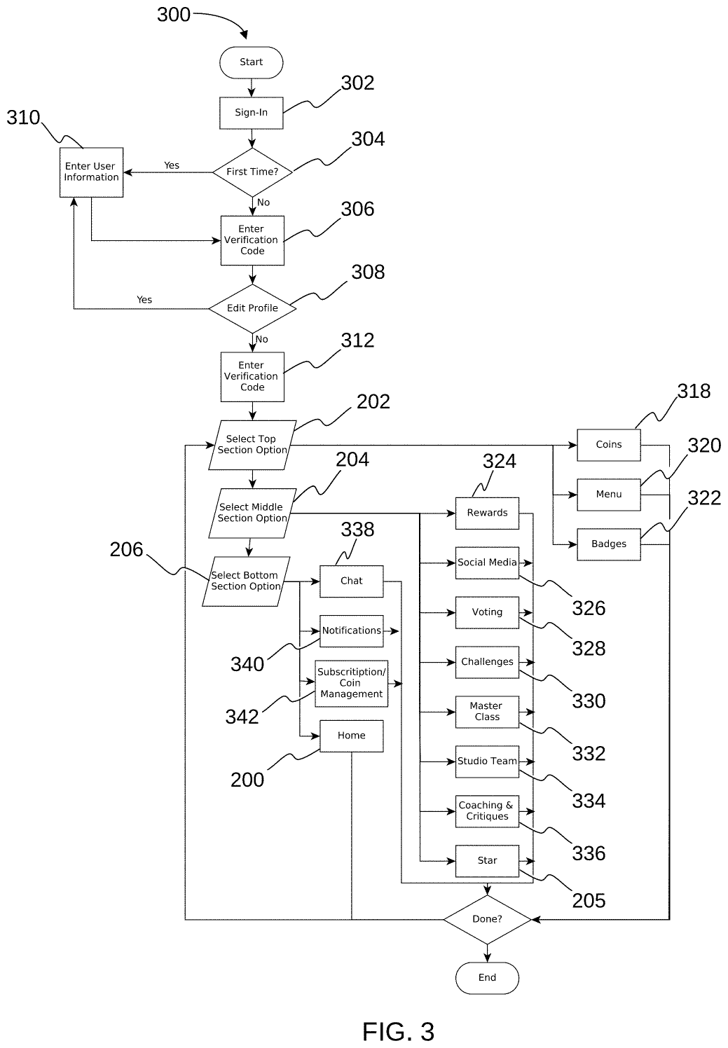

Referring now to FIG. 3, there is shown a flowchart diagram 300 of some steps of a method for the system 100. As can be seen, the flowchart 300 comprises all the modules found on the participant's home screen shown in FIG. 2. First, the participant signs 302 into the system 100. The a determination is made whether or not this is the participant's first time 304 entering the system 100. If the participant has never used the system 100 before, they are sent to the user information module 310 to create an account. Here the participant enters basic information: First, last name, birthdate and gender. They also identify themselves by participant type—For example, Dancer, Dance Parent, Studio Teacher and Studio Owner. Allowing the engagement to be more customized to the participant. Once the user is entered into the system 100, a verification module 306 either sends a verification number, password or other identifier to the participant for security and to insure that only authorized participants enter the system. Then, the participant enters the verification code 306 to gain access to the system. If the participant wants to edit any portion of their profile, an option is presented on the home screen to edit the profile 308 of the participant. Then, the home screen 200 is presented to the participant. Next, the participant can select from the top 202, middle 204 or bottom 206 section icons to access various modules of the system 100.

The top section 202 icons provide the participant with access to a coins module 318, a menu module 320 and a badges module 322. Participants can purchase or upgrade a subscription, and/or buy more coins. Participants can earn additional coins by watching videos or filling out surveys.

The menu module 320, comprises the following options for the participants to select from:
a. Social media profile—participants can update profile, change their profile picture and bio, review gems and badges, add friends, review the main wall and upload media
b. Manage subscription—participants can review and change subscription package.
c. User history—This tracks participant's coin purchases, for example, dance gear, certification courses, critiques, etc.
d. Invite friends—participants can invite friends, teachers and studio owner to participate.
e. Settings—Manage password, turn on/off notifications, account information transaction detail and delete account.
f. Legal—Contains the terms and conditions and privacy policy
g. About us—Is an overview of the company and its mission
h. Contact us—Sends an email to contact the company office
i. Logout—Participants can log out of the app.

The middle section 204 provides the participant with access to a badge module 322, where participants can review the badges they have earned. The middle section 204 icons also provide the participant with access to a rewards module 324, a social media module 326, a voting module 328, a challenges module 330, a master class module 332, a studio team module 334, a coaching and critiques module 336 and the star module 205. The rewards module 324 allows participants to use their coins to purchase items and claim prizes. Each item in the rewards module 324 is given a specific coin value for the participant to select from six categories comprising:
a. coaching—participants can purchase one on one consultation with certified coaches
b. critiques—participants can purchase critiques to upload their video for professional feedback
c. dance challenges—participants can select from a list of professionals to join a dance challenge of their choice.
d. master classes—participants can participate in the certification courses
e. gear—participants can purchase clothing types including but not limited to tops, pants, shorts, hoodies, hats, accessories, etc.
f. advanced features—participants can purchase a spin the wheel, refill votes, and send customized compliment.

The social media module 326 (instagram like social postings) provides participants an social area where they can post text, pictures and videos. Participants can also like posts, pictures and videos by other participants, comment on posts, pictures and videos. Participants can also add friends, flag inappropriate content. If a participant has earned a badge, it will posted by the system 100 for viewing. Participants can also upload their virtual-battle video for public viewing.

The voting module 328 lets participants vote for their favorite challenge entries. Participants can search for other participants, or use the filter to search more specifically. The participants in the system 100 community can view the current video challenges and vote for their favorite. Each month, the most voted videos are sent to professional affiliates, giving the participant a head start to their career.

The challenges module 330 is where participants learn, film and upload the activity challenges and post to the social media module 324 and the voting module 328. Each participant goes through a three step process. First, class challenge, participants select from a list of professional teachers, and learn the activity, film themselves performing the activity, and upload to the system 100. Participants are then given a choice to use coins for a professional critique. This can be instrumental in the speed of how the participant picks up material, growth and knowledge. The second challenge is the next step, where the participant adds their own creative movement to what they already learned. They film themselves and upload it to the system 100. They're given a choice to use coins for a professional critique. This exercise is a way for the participant to improve their adaptability of movement and build skill. The final challenge is a concept challenge. The participant will be given different levels to this challenge to enhance their capacity of production work. Participants film themselves, upload the video to the system 100 and are given a choice to use coins for a professional critique. At the end of this challenge, they can choose to have their video privately or publicly viewed and posted onto the voting module 328.

The master class module 332 is the main hub of the certification courses/video library/conditioning. The certification courses are broken down into topics, then classes to support the topic, followed by a quiz. At the end of the quiz, participants are awarded a badge and gems. Each badge that participants are awarded are automatically posted on the homepage 204, on their social media 326, and their gems will give them a new position on the leaderboard.

The studio team module 334 once an owner of a group subscribes to the studio team program, the owner can sign up individuals of their group to participate in competitive group learning. These courses are set up, based on user type like dancer or dance parent. The certification courses are structured like the master class module 332. Each user group will have access to their own page containing their specific course, topics, classes and quizzes. At the top of the users group page, there is a monthly countdown shown for a live questions and answers with a professional affiliate. The owner of the group, will have a separate page only available to the owner and assigned administrators. At the top of this page the owner will have a bi-monthly countdown for a live roundtable discussion with other group owners to discuss pertinent issues shared by this community. The session is lead by a professional affiliate. These sessions are recorded and archived on the bottom of the page to be viewed at the owners discretion.

The studio owner page control panel also comprises instructions in the system 100 to:

1. invite participants

2. manage groups and courses

3. track user progress

4. schedule courses, topics and classes

5. broadcast announcements

Course buttons are displayed on the bottom of the page. Additional programs like assistant teaching training course can be added to this module. The additional courses will continue to add value to the owner's business.

The coaching and critiques module 336 gives participants access to professional coaching, that is instrumental in the growth and direction of the participants. After the participant signs up for a sixty minute one on one session with a selected professional, at a time that works for both the professional and the participant, the coaching session is held. The coaching sessions are done live on via the Internet. Once the session is complete, the video of the session along with notes and homework are upload to the participants account to view at their discretion. Participants can keep or delete the videos.

Professional critiques are available for participants in the following categories, solo, class challenge, next step challenge, virtual-battle challenge. These critiques are uploaded by the participants. The system 100 will send the videos to a professional certified member of the staff to give constructive feedback. Once the critique is complete, it will be uploaded onto the participant's exclusive account to view at their own discretion. If a group owner has signed up, the group owner is given a separate group critique to improve group performance.

The star module 205, includes social media members search. Featured winners of the dance challenges will be highlighted each month. The gem leaderboard decides the featured members of each month. Our associated sponsors allows for the users to learn more about their company.

The bottom section 206 icons provides the participant with access to return to the home screen 200, a chat module 338, where users can chat with one another, form groups and video conference, a notifications module 340, where admin notifications are sent, and a subscription/coins management module 342, which takes them to a subscription page.

What has been described is a gamified social media educational system 100 for both online and in person training for participants to take control of their lives that overcomes the limitations of the prior art. Although the present invention has been described with a degree of particularity, it is understood that the present disclosure has been made by way of example and that other versions are possible. As various changes could be made in the above description without departing from the scope of the invention, it is intended that all matter contained in the above description or shown in the accompanying drawings shall be illustrative and not used in a limiting sense. The spirit and scope of the appended claims should not be limited to the description of the preferred versions contained in this disclosure.

All features disclosed in the specification, including the claims, abstracts, and drawings, and all the steps in any method or process disclosed, may be combined in any combination, except combinations where at least some of such features and/or steps are mutually exclusive. Each feature disclosed in the specification, including the claims, abstract, and drawings, can be replaced by alternative features serving the same, equivalent or similar purpose, unless expressly stated otherwise. Thus, unless expressly stated otherwise, each feature disclosed is one example only of a generic series of equivalent or similar features.

Any element in a claim that does not explicitly state “means” for performing a specified function or “step” for performing a specified function should not be interpreted as a “means” or “step” clause as specified in 35 U.S.C. § 112.

What is claimed is:

1. A computer implemented method for a gamified social media educational platform, the method comprising:
a) executing instructions on a server with at least one processor and at least one storage for interacting with a participant via the Internet using four distinct educational modules, the educational modules comprising:
1) an introduction module for focus and commitment;
2) a setting goals module for long term, flexible direction in life;
3) a success module for harnessing a participant's passion; and
4) a tools of the trade module for mental, emotional, physical tools.

2. The method of claim 1, wherein the introduction module comprises instructions presented to the participant:
a) teaching basic habits required of every participant to maximize and elevate the experience and improve the end result; and
b) teaching fundamentals values need to complete the course.

3. The method of claim 1, wherein the introduction module comprises instructions presented to the participant for:
a) determining a value of long-term goals that have been set;
b) determining a value of short-term goals that have been set;
c) determining a reason to visualize the long-term and short-term goals;
d) researching the long-term and short-term goals;
e) organizing the long-term and short-term goals for the individual participant;
f) determining a follow through plan, so that the participant continues to work; and
g) displaying a workbook with the modules, where a participant builds a vision board to express the participant's ideal future.

4. The method of claim 3, wherein the participant is shown compiled professional research to assist with the step of researching.

5. The method of claim 1, wherein the success module comprises instructions presented to the participant:
a) presenting daily steps for success, where each step is one day of an activity;
b) determining investment in self;
c) expressing by other professionals in the field using personal examples so the participant can determine their connection to the activity;
d) determining emotional connection to the activity;
e) determining the participant's inspiration for the activity and how to stay inspired;
f) teaching the participant how to understand the “why” in the activity, when there is a passion driving the activity; and
g) filling out a daily journal of exercises provide by the system 100 to assist in maintaining the participant's goal that were previously determined.

6. The method of claim 1, wherein the tools of the trade module comprises instructions presented to the participant:
a) building the participant's self worth by introducing mental, physical and emotional tools for a participant to master;
b) determining mental tools to assist the participant with the activity;
c) determining physical tools, the building blocks of the activity;
d) determining any advanced physical tools for the activity;
e) emotional tools for interconnecting the participant's inner self and external connection to the activity;
f) determining the participant's attitude and dealing with failure;
g) providing a digital workbook with specific exercises for each of these steps, showing participants how to sharpen their unique skills and mindset, in order to achieve their long-term goals.

7. The method of claim 6, wherein the tools of the trade module further comprises the step of addressing the mental, emotional and physical skills of the specific task the participant is pursuing.

8. The system of claim 1, wherein the method further comprises instructions executable on a processor for introduction module comprises instructions presented to the participant for:
a) determining studio activity sessions for an at home conditioning course;
b) determining healthy living for mental and physical health;
c) determining activity life training etiquette; and
d) determining beyond the activity professions and educational opportunities based upon the participant's goal.

9. The method of claim 6, wherein selecting the physical exercises for the participant for the activity sessions module comprises a deeper explanation of the level of conditioning needed by the participants, to keep their body safe.

10. The method of claim 8, wherein the healthy living module comprises the steps of:
a) demonstrating physical injury prevention;
b) determining nutrition for the participant; and
c) evaluating mental health of the participant.

11. The method of claim 8, wherein the activity life module comprises the steps of:
a) demonstrating activity etiquette;
b) demonstrating communication between participant and parent;
c) communicating with teachers and studio owners by the participant;
d) finding a life, school and activity balance;
e) providing methods for dealing with the anxiety;
f) providing expectation analysis for rehearsals and practice for the participant to achieve the best results; and
g) providing expectation analysis for getting the most out of performance for an activity.

12. The method of claim 8, wherein the beyond the activity module comprises the steps of:
a) determining by the participant professional aspirations;
b) determining by the participant educational aspirations;
c) determining by the participant continued education; and
d) determining by the participant an overall understanding of what the participant has to offer beyond school.

13. A computer enabled gamified social media educational platform system comprising:
a) a server comprising:
1) at least one processor providing access to an educational platform;
2) a plurality of executable educational module instructions from a first storage medium;
3) instructions for reading participant information; and
4) storage for storing the participant information, including: progress, scoring, bonuses, coins, badges, gems and other relevant data of the participant in the storage medium;
b) a plurality of participant electronic devices communicatively coupled, via Internet, to the server, each participant electronic device associated a plurality of participants; and
c) a second storage operably connected to the server, wherein gamification instructions for learning executable on the server and on the plurality of participant electronic devices to motivate participants and to control interactions between the plurality of participants and a social media educational platform using their respective electronic devices, wherein the respective electronic devices can display:
1) an introduction module for focus and commitment;
2) a setting goals module for long term, flexible direction in life;
3) a success module for harnessing a participant's passion;
4) a tools of the trade module for mental, emotional, physical tools; and
5) an activity sessions module for selecting physical exercises for the participant.

14. The system of claim 13, wherein the gamified social media educational platform comprises:
a) certification courses for teachers, participants and parents;
b) private coaching, wherein certified professionals give sixty minute coaching sessions to participants;
c) professional critiques, wherein a participant can upload a video of themselves performing an activity from a class or the activity that is analyzed by certified staff, and the video is returned to the participant with comments, suggestions and where to focus for the participant to achieve the greatest improvement; and
d) classes taught by professionals in the industry.

15. The system of claim 14, further compromising a) participants pay coins earned using the system 100 for the class, learn the from the class, then film themselves doing the activity that was taught;
b) videos will be entered into a contest, where winners get more coins and professional critiques are available;
c) a virtual-battle module will be available for videos entered in step b;
d) other participants vote on the virtual-battle videos to determine a winner; and
e) additional benefits are provided to both the winner and the other participants of the virtual-battle.

16. The system of claim 14, wherein the coaching sessions are also available online.

17. The system of claim 13, wherein the gamified social media educational platform further comprises:
a) an activity wall module that provides participant's personal social media type wall;
b) a group creation module, where a participant uses coins to create a group, join a group or both create and join a group;
c) a list of active courses module to lead the participant back to the learning platform;
d) a messaging and notifications module for communication from participants and staff;
e) a real time chat module including video group conferencing;
f) a friends/followers/following module that provides those social media functions;
g) a coins module that tracks earned for daily logins, posts, and general activity within the system, wherein coins can be used to buy prizes in a reward center and can be purchased by signing up for a silver, gold, or platinum level monthly membership;
h) a compliments module, wherein compliments can be sent by each participant;
i) a commerce module, wherein participants pay to access another participants profile, and wherein a percentage of the sale is given to the participant who created the profile;
j) a media module for that organizes all participant uploaded media; and
k) a vendor module for selling gear related to the activity.

18. The system of claim 13, wherein the gamified social media educational platform further comprises:
a) a reward center module, wherein coins earned by participants can be exchanged for other items, such as, groups, sending compliments, advanced social media features, classes, videos, certification courses, and gear.
b) a coins module, wherein participants earn coins by being involved in the system 100's social media, or by purchasing a membership level of silver, gold, or platinum membership, wherein coins are also awarded when a participant watches select videos, for taking surveys and winning contests;
c) a badges module, wherein when participants complete a purchase, watch a class, submit a video or submit homework, they earn a badge for their profile that is displayed on the participant's social media wall; and
d) a gems module, wherein each badge earned by a participant is worth ten gems. at the end of the month, the top ten gem earners win and get featured as “featured participant” on the membership site, increasing their exposure, wherein participants also earn gems when they receive a “compliment” from other participants.

19. The system of claim 17, wherein the rewards module, provides participants with coins, badges and gems for getting involved in the social community, where the coins are redeemable for activity related merchandise, including videos of the teachings, classes, gear and additional social media perks such as video chats.

20. The system of claim 13, wherein the gamified social media educational platform further comprises a virtual-battle module that is a unique online competition between participants, the virtual-battle module comprising:
1) once an activity is learned in a class, the participant is prompted to record and download themselves performing the activity, which advances the participant to the next level;
2) “the next step” wherein the participant creates the next activity and uploads it to the system and wherein a professional critique is offered for the participant's video;
3) once uploaded, the participant is now invited to a secret part of the system called “virtual-battle” where participant are given levels of video challenges to upload and compete with other participants and wherein various prizes are awarded to the winner of each virtual-battle;
4) virtual-battle levels comprise participation with friends, at unique locations and/or special effects in editing software;
5) voting, wherein participants vote for their favorite in each virtual-battle and earn coins for voting; and
6) gems, wherein the participant with the most gems at the end of the month is featured on the main webpage, wherein gems are earned from badges in the learning center, or compliments in the social media platform.

21. The system of claim 13, wherein the gamified social media educational platform further comprises a studio team module, wherein participants sign up for an activity group to participate in competitive group learning, wherein an owner of the activity group, has a control panel with executable instructions to:
a) invite participants;
b) manage groups and courses;
c) track participant progress;
d) schedule courses, topics and classes;
e) broadcast announcements;
f) archive round table discussions held with other group owners; and
g) a courses button that accesses:
1) participant certification;
2) parent certification; and
3) assistant teaching training courses.

22. The system of claim 13, wherein the gamified social media educational platform further comprises a parent certification course module comprising:
a) providing a video course with lessons, wherein the lessons are divided into smaller video modules comprising at least:
1) Introduction to the certification course;
2) focus on success—understanding a different mindset than your used to focus on success vs. focus on things like the trophy;
3) Finding Balance—Understanding the investment, the time and money spent on an activity, and all sacrifices made by parents and participants;
4) Injury Prevention—keeping your child safe;
5) Communication—with a child, with your spouse, with your studio owner. Importance of, how to, what to say, leaving emotions out;
6) Supercharge Success—driving their child's passion for activity to greatness by harnessing passion;
7) Performance Anxiety—dealing with parent anxiety/participant anxiety and how to overcome it;
8) Self-worth—Building self-worth with the short term and long-term goals set, and understanding the value for the long term in each short term goal;
9) Accountability—staying true to yourself first, staying true to your word, and your commitment;
10) 5, 6, 7, 8 to Success—creating their child's next step for their journey in the activity and how to use the lessons learned throughout the series to harness success in the activity, and in life; and
11) Bonus lessons comprising training in social media, body image, college options, professional options, bullying, healthy living, and preparing for auditions.
